(19) United States
(12) Patent Application Publication
(10) Pub. No.: US 2010/0049233 A1
(43) Pub. Date: Feb. 25, 2010

(54) LANCET HAVING INTEGRATED DRIVE MECHANISM

(75) Inventors: Maiya Shur, Reston, VA (US); Kevin Young, Boston, MA (US); Gaurav Rohatgi, Franklin, MA (US); Damien Vizcarra, Pasadena, CA (US)

Correspondence Address: YOUNG BASILE, 3001 WEST BIG BEAVER ROAD, SUITE 624, TROY, MI, 48084, US

(73) Assignee: ABBOTT DIABETES CARE INC., Alameda, CA (US)

(21) Appl. No.: 12/197,618

(22) Filed: Aug. 25, 2008

(51) Int. Cl. A61B 17/34

(52) U.S. Cl. 606/182

(57) ABSTRACT

A lancet for use with a lancing device is provided. The lancet includes a lancet body and a drive mechanism attached to the lancet body. A needle projects from an end of the lancet body. The drive mechanism is operative in response to insertion of the lancet in the lancing device to bias the lancet toward a pricking position.

### FIELD OF THE INVENTION

The present invention relates to the field of lancing systems, and more particularly to lancets.

### BACKGROUND

Lancing systems are typically handheld units that permit users to draw blood for testing and diagnostic purposes. These systems can include a housing with a piercing aperture. The housing can contain a firing mechanism, typically a spring, permanently attached to the housing. A lancet holding a needle can be sized for insertion into the housing through the piercing aperture.

In operation, a user can insert the lancet in the housing, cock the firing mechanism, and then place the lancing system against his skin. With the lancing system against his skin, the user can actuate the firing mechanism to propel the lancet, thereby causing the needle to puncture his skin. The lancet can be disposed, a second lancet can be inserted into the housing, and the lancing process can be repeated with the same firing mechanism.

### SUMMARY

Lancets for use with lancing devices, as well as methods of using a lancet, are provided. In accordance with one example, a lancet for use with a lancing device includes a lancet body. A drive mechanism is attached to the lancet body, and a needle projects from an end of the lancet body. The drive mechanism is operative in response to insertion of the lancet in the lancing device to bias the lancet toward a pricking position.

In another example, a lancing system includes a lancing device defining a lancet receptacle. A lancet is slidably received in the lancet receptacle, and the lancet includes a lancet body having a drive mechanism attached thereto. A needle projects from an end of the lancet body. The drive mechanism is operative in response to insertion of the lancet in the lancet receptacle to bias the lancet toward a pricking position.

In still another example, a method of using a lancing device and a separate lancet having a needle is described. The method includes inserting the lancet into the lancing device to energize a resilient portion of the lancet. The method also includes releasing the lancet to allow the lancet to move under the urging of the resilient portion toward a pricking position.

### BRIEF DESCRIPTION OF THE DRAWINGS

The description herein makes reference to the accompanying drawings wherein like reference numerals refer to like parts throughout the several views, and wherein:

FIG. 1 is an exploded view of an example of a lancing system;

FIG. 2 is a cross sectional view of the lancing system of FIG. 1 along line A-A;

FIG. 3 is a cross sectional view of the lancing system of FIG. 1 along line A-A when the lancet is partially inserted into the lancing device;

FIG. 4 is a cross sectional view of the lancing system of FIG. 1 along line A-A when the lancet is inserted further into the lancing device than shown in FIG. 3;

FIG. 5 is a cross sectional view of the lancing system of FIG. 1 along line A-A when the lancet is in a retained position;

FIG. 6 is a cross sectional view of the lancing system of FIG. 1 along line A-A when the lancet is in a pricking position;

FIG. 7 is a cross sectional view of the lancing system of FIG. 1 along line A-A with the lancet in a retracted;

FIG. 8 is a perspective view of another example of a lancet;

FIG. 9 is a perspective view of yet another example of a lancet;

FIG. 10 is an exploded cross sectional view of another example of a lancing system;

FIG. 11 is another example of a lancet; and

FIG. 12 is yet another example of a lancet.

### DETAILED DESCRIPTION

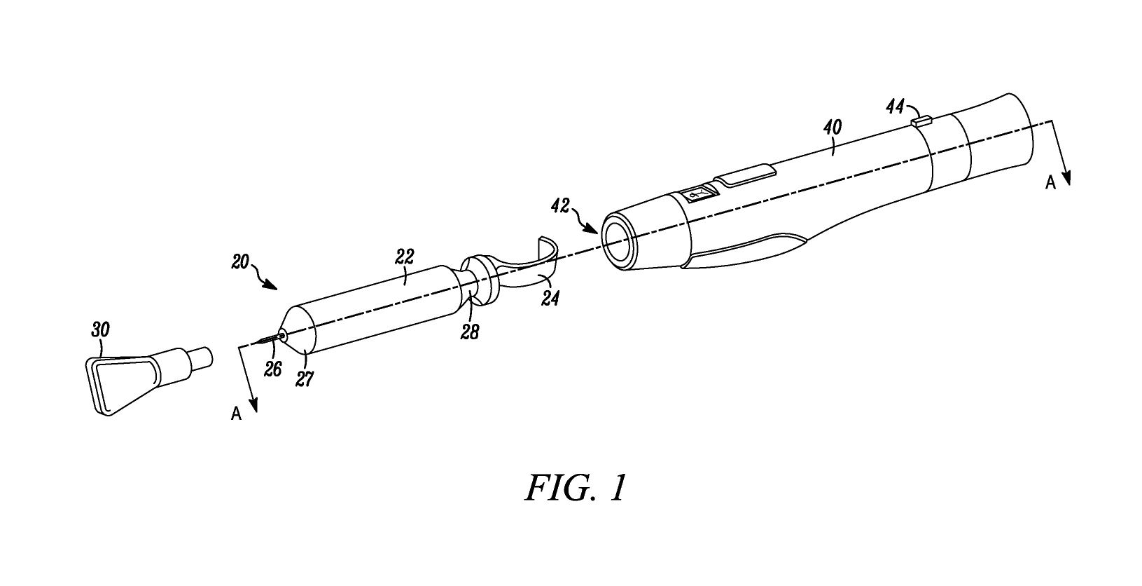

FIGS. 1-12 illustrate various lancets according to the invention. As shown in FIG. 1, a lancing system 10 includes a lancet 20, a sleeve 30, and a lancing device 40.

As shown FIG. 1, the lancet 20 includes a lancet body 22, a drive mechanism 24, and a needle 26. The lancet 20 may be disposable, with a new lancet 20 used for each operation. The lancet body 22 as illustrated is generally cylindrical with the exception of a tapered end 27 and a notch 28. However, the lancet body 22 can have a different shape with a rectangular, oval, or other polygonal cross section. The shape of the lancing body 22 can vary along its length by, as an example, transitioning from a triangular front portion to a cylindrical rear portion.

Further, the shape of the lancet body 22 can be designed to provide an operational advantage. For example, the lancet body 22 can have an asymmetrical cross section that is keyed to the lancing device 40 to require the lancet 20 be correctly oriented for insertion into the lancing device 40, or the lancet body 22 can be ergonomically shaped for comfortable and safe handling.

The tapered end 27 of the lancet body 22 can provide a transition between the cylindrical portion of the lancet body 22 and the needle 26. While illustrated as conically shaped, the end 27 of the lancet body 22 can have a different shape. For example, the tapered end 27 can define a groove circumscribing the lancet 20 to allow the sleeve 30 to be snap-fit to the lancet 20. As another example, the end 27 need not be tapered, as the lancet body 22 can be cylindrically shaped instead of conically shaped at the end 27.

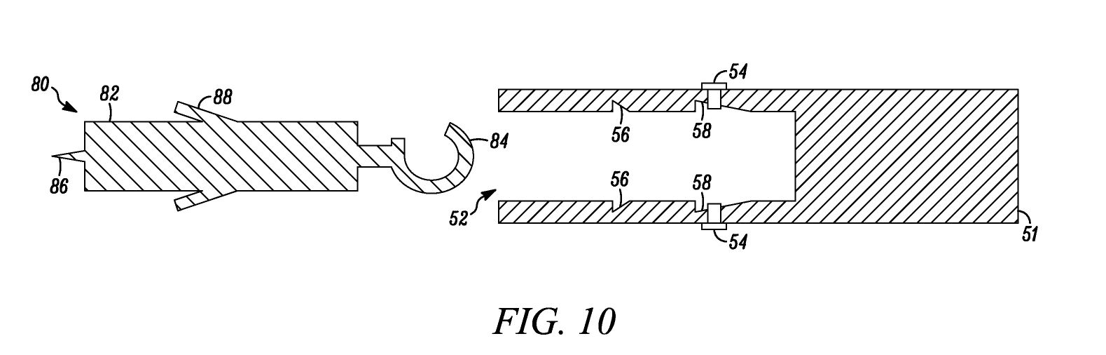

The notch 28 can be shaped for engagement with the lancing device 40. The notch 28 illustrated in FIG. 1 is generally V-shaped and circumscribes the lancet body 22. However, the geometry of the notch 28 can depend on the type of lancet holder included in the lancing device 40, and alternatively shaped notches can be included. For example, an alternative notch can extend around only a portion of the circumference of the lancet body 22, and multiple notches of this type can be spaced around the circumference of the lancet body 22. Further, the notch 28 can be positioned at a different location on the lancet body 22 from the position as illustrated in FIG. 1, and multiple notches 28 can be formed along the length of the lancet body 22 to allow the lancing device 40 to secure the lancet 20 at various positions. Some lancing devices include lancet holders that do not require the notch 28 to be formed in the lancet 20. Thus, the lancing body 22 can include another structure for engagement with the lancing device 40, such as wings 88 (discussed below in reference to FIG. 10) or bosses projecting from the lancet body 22.

As shown in FIG. 1, the drive mechanism 24 has a hooked or C-shape and is integrally formed with the lancet body 22. However, drive mechanisms having different structures than the drive mechanism 24 can alternatively be used, as a drive mechanism can be any structure attached to a lancet that is operative in response to insertion of the lancet in a lancing device to bias the lancet toward a pricking position (i.e., a position in which a needle of a lancet engaged with a lancing device can contact the skin of a user). For example, a drive mechanism can be a resilient structure that is energized (i.e., that stores potential energy) when deformed to bias the lancet toward the pricking position. As another example, a drive mechanism can be chamber containing pressurized air attached to a lancet. The chamber can be pierced when the lancet is inserted in a lancing device to release the pressurized air from the chamber, thereby biasing the lancet toward the pricking position.

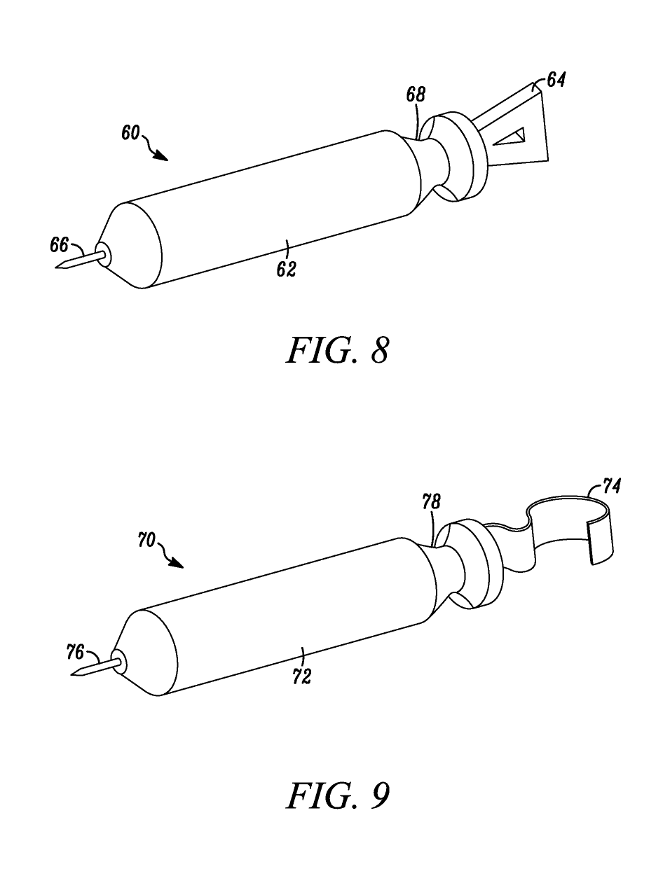

Referring again to the lancet 20 shown in FIG. 1, the shape of the drive mechanism 24 can be a factor of the mechanical properties of the material from which the drive mechanism 24 is constructed, the shape of the lancet body 22, the geometry of the lancing device 40, and the desired driving force, among other considerations. For example, the drive mechanisms 64 and 74 discussed in reference to FIGS. 8 and 9, respectively, are examples of drive mechanisms having different shapes than the drive mechanism 24 illustrated in FIG. 1.

As mentioned above, the lancet body 22 and drive mechanism 24 can be integrally formed. For example, the lancet 20 can be molded to form a monolithic body in the shape of the lancet body 22 combined with the drive mechanism 24. The lancet 20 can be formed from plastic, another polymer, a composite, or another material. The specific material used can depend on a variety of factors, such as the geometries of the lancet 20 and lancing device 40, the desired driving force applied to the lancet 20, the sharpness and strength of the needle 26, and the desired skin puncture depth.

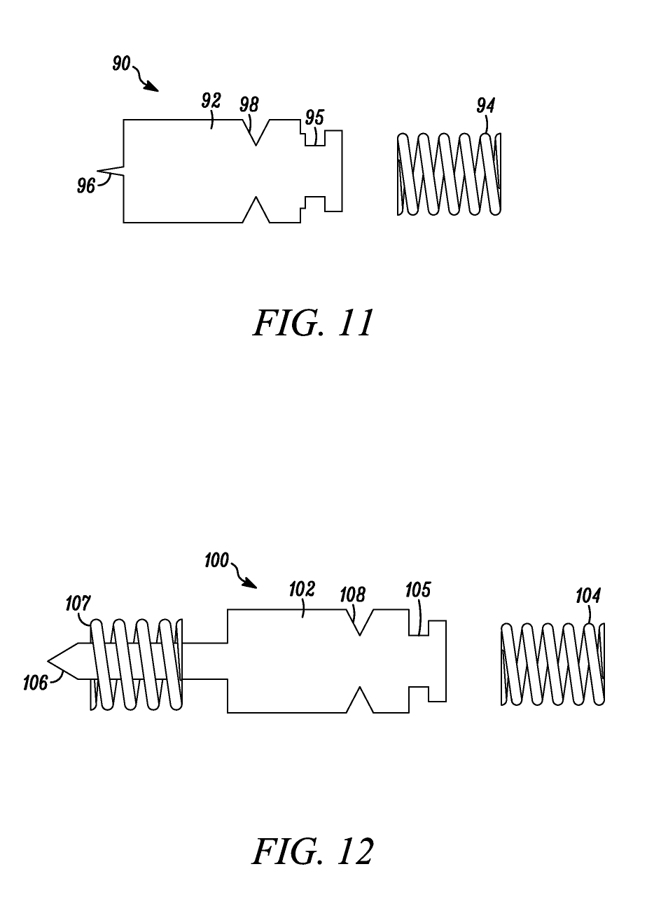

Also, while the drive mechanism 24 can be formed integrally with the lancet body 22 as shown in FIG. 1, the drive mechanism 24 and lancet 22 can alternatively be separate pieces attached after being formed as is discussed below in greater detail. For example, drive mechanisms 94 and 100 discussed in reference to FIGS. 11 and 12, respectively, are examples of separate drive mechanisms.

The needle 26 can project from the lancing body 22 as shown in FIG. 1 to extend along a longitudinal axis of the lancet body 22. The distance the needle 26 projects from the lancet body 22 can be predetermined such that the needle 26 can puncture the skin of a user to a sufficient depth to draw blood, but a small enough depth such that the pain the user experiences is low and the user can quickly heal. The term “needle” encompasses any sharp tip designed to puncture the skin of a user to draw blood, and the needle 26 can be made from metal (e.g., steel) or another material. For example, the needle 26 can be a polymer molded integrally with the lancet body 22, though the needle 26 can be attached to the lancet body 22 in other ways. One other way of attaching the needle 26 to the lancet body 22 includes placing the needle 26 in a mold used for forming the lancet body 22 prior to the forming the lancet body 22, then molding a polymer around the needle 26 to secure the needle 26 to the lancet body 22. Alternatively, the needle 26 can include an integral attachment structure that snaps onto the lancet body 22, or the needle 26 can include a threaded distal end that is screwed into the lancet body 22. As another example, the needle 26 can be press fit into the lancet body 22.

The sleeve 30 illustrated in FIG. 1 can be engaged with the lancet 20 to cover the needle 26. The coupling between the sleeve 30 and the lancet 20 can be, for example, a friction or snap fit. The sleeve 30 can be coupled to the lancet 20 prior to use of the lancet 20, and a user can remove the sleeve 30 after fully inserting the lancet 20 into the lancing device 40. Alternatively, a different structure from the sleeve 30 illustrated in FIG. 1 can be used to cover the needle 26. For example, a lancet can be molded to include an integral shield covering three sides of a needle and extending past the tip of the needle, and the shield can be perforated or otherwise designed to break away from the lancet if bent away from the needle.

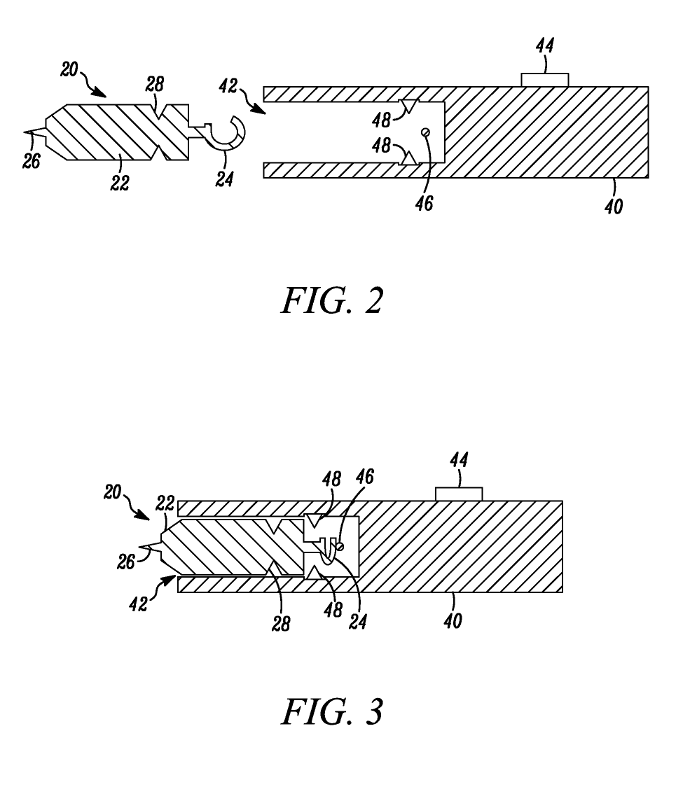

Also shown in FIG. 1, the lancing device 40 can define a lancet receptacle 42, and the device 40 can include a trigger 44. As shown in FIGS. 2-7, the lancet receptacle 42 can be sized to slidably receive the lancet 20. While the illustrated lancet receptacle 42 is a cylindrical bore, the receptacle 42 can be any bore, chamber, passage, guide, track or other structure designed to receive the lancet 20. As such, the receptacle 42 can have a different shape than illustrated. For example, the receptacle 42 can be keyed to the shape of the lancet 20, as mentioned above, to ensure a unique orientation of the lancet 20 within the lancing device 40. Alternatively, a lancet receptacle can be accessible through the side of a lancing device instead of through the longitudinal end of the lancing device 42 of FIG. 1.

The lancing device 40 can include a latch 46 designed to limit the motion of the lancet 20. The latch 46 as illustrated is rod-shaped and extends across the receptacle 42. However, the latch 46 can be any structure of the lancing device 40 that limits the travel of the lancet 20 past the pricking position. For example, the latch 46 can have an alternative shape, such as a ring shape. The position of the latch 46 can be set to permit a predetermined amount of movement of the lancet 20, and can therefore be based on the shape and physical properties of the lancet 20, among other considerations. Additionally, the lancing device 40 can be formed without a latch 46.

The trigger 44 can be in communication with a lancet holder, shown in FIG. 2 as a pair of moveable bosses 48. While the trigger 44 as illustrated is configured as a single button is illustrated, a trigger can be any structure in communication with a lancet holder to selectively disengage the lancet holder from a lancet. For example, a rod inserted through apertures in the lancing device and lancet to hold the lancet in the lancing device can function as a trigger, as removing the rod disengages the lancet from the lancing device. Additionally, depending on the design of a lancet and lancet body, a trigger may not be necessary.

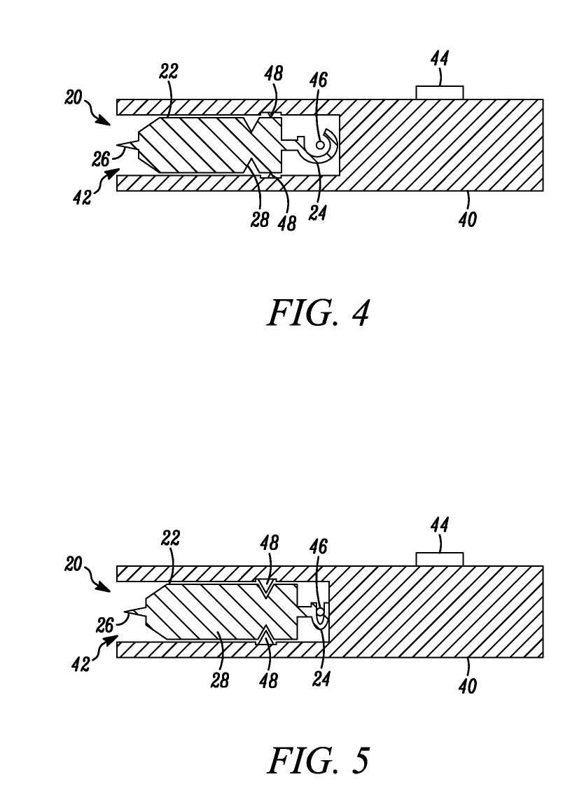

The lancet holder shown in FIG. 2 includes generally V-shaped moveable bosses 48, with the V-shape designed to correspond with the generally V-shaped notch 28 in the lancet 20. However, other bosses can have alternative geometries sized and shaped to engage lancet bodies of different shapes than the body 22. The moveable bosses 48 can have a normal position shown in FIG. 2 extending radially into the receptacle 42, and the bosses 48 be moved radially outward from the normal position when a force is applied to the bosses 48 in a radially outward direction as shown in FIG. 4. A spring in the lancing device 40 can bias the bosses 48 toward the normal position.

Actuation of the trigger 44 can be communicated to the moveable bosses 48, for example, the trigger 44 can be physically linked to the bosses 48, to move the bosses 48 radially outward from their normal position as shown in FIG. 2. While the lancet holder is shown as moveable bosses 48, the lancet holder can be any structure that can selectively hold the lancet 20 in a retained position (i.e., a static position in the lancing device 40). That is, different lancet holders from the moveable bosses 48 can be used, such as the indentations 56 and 58 described in reference to FIG. 10 or the rod inserted through apertures in a lancet and lancet device as mentioned above. Additionally, depending on the design of a lancet and lancet body, a lancet holder may not be necessary.

FIGS. 2-7 illustrate the lancing system 10 in operation. The lancet 20 as shown in FIG. 2 is disengaged from the lancing device 40, as the lancet 20 in FIG. 2 is shown prior to inserting the lancet 20 in the lancing device 40. Since the trigger 44 is not actuated, the moveable bosses 48 are in their normal position extending into the receptacle 42 (i.e., positioned to engage the lancet 20).

FIG. 3 shows the lancet 20 during insertion into the lancing device 40. The drive mechanism 24 is deflected as it contacts the latch 46. Since the drive mechanism 24 can be resilient, the drive mechanism 24 can produce a force in the axial direction of the lancing device 40 when deflected as shown in FIG. 3. A user can overcome the force produced by the drive mechanism 24 by urging the lancet 20 further into the lancing device 40 to the position shown in FIG. 4, further deflecting the drive mechanism 24 in the process. The moveable bosses 48 remain in their normal position with the lancet 20 positioned as shown in FIG. 3.

FIG. 4 shows the lancet 20 at a later stage of insertion compared to FIG. 3. Axial movement of the lancet 20 into the lancet receptacle 42 from the position shown in FIG. 3 to the position shown in FIG. 4 has caused the lancet 20 to apply a force to the bosses 48. Due to the V-shape of the bosses 48, a component of this force acts on the bosses 48 in a radially outward direction, biasing the bosses 48 radially outward of their normal position to allow insertion of lancet 20 past the bosses 48.

Additionally, at a point between the positions shown in FIGS. 3 and 4, the drive mechanism 24 can be sufficiently deflected that it produces a large enough force that the deflected portion of the drive mechanism 24 as shown in FIG. 3 rebounds around the latch 46 to the position shown in FIG. 4. That is, the drive mechanism 24 can return to its normal shape due to its resiliency. As a result, if a user continues to urge the lancet 20 into the lancing device 40 from the position shown in FIG. 3, the drive mechanism 24 can spring around the latch 46 back to its normal shape.

FIG. 5 shows the lancet 20 at a later stage of insertion compared to FIG. 4, with the lancet 20 in the retained position. The lancet 20 is positioned such that the moveable bosses 48 are aligned with the notch 28 defined by the lancet 20, allowing the moveable bosses 48 to be biased back toward their normal position to engage the notch 28. Additionally, the drive mechanism 24 is deflected due to contact with the lancing device 40. Due to its deflection, the drive mechanism 24 is energized and produces a force in the axial direction of the lancing device 40; i.e., a force biasing the drive mechanism 24 toward the pricking position. However, the force produced by the drive mechanism 24 can be less than a force required to bias the bosses 48 radially outward to disengage the lancet 20 from the bosses 48. As a result, the lancet 20 can be held in the retained position shown in FIG. 5 despite the force toward the pricking position produced by the drive mechanism 24.

Also, in the retained position as shown in FIG. 5, the needle 26 can be entirely within the lancing device 40. The position of the needle 26 can prevent accidental contact of the user with the needle 26. While the sleeve 30 may be removable regardless of the position of the lancet 20, the sleeve 30 can be removed once the lancet 20 is in the retained position. By waiting until the needle 26 is entirely within the lancing device and the lancet 20 is in the retained position to detach the sleeve 30 from the lancet 20, the likelihood of accidental contact between the needle 26 and the user can be reduced.

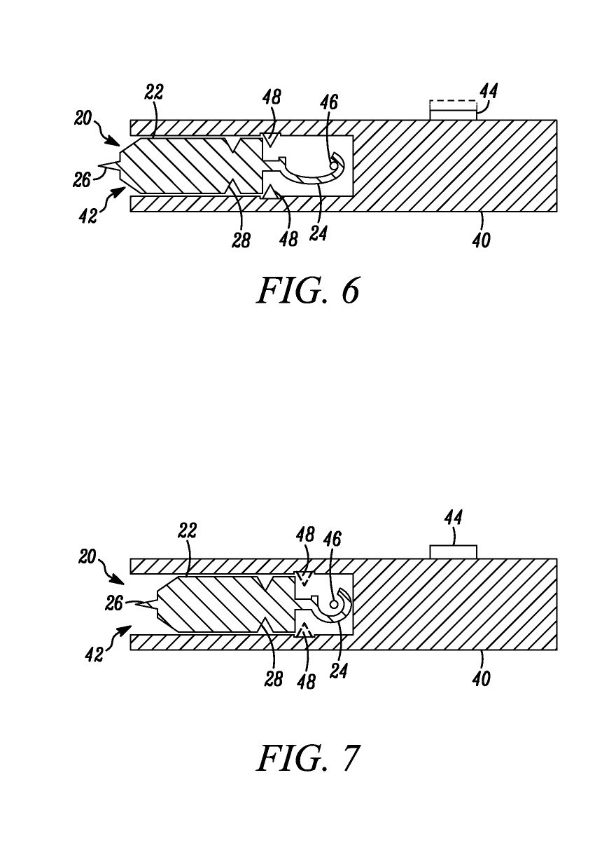

FIG. 6 shows the lancet 20 after the trigger 44 has been actuated. Actuation of the trigger 44 can move the bosses 48 radially outward from their normal position, thereby disengaging the bosses 48 from the lancet 20. With the bosses 48 disengaged from the lancet 20, the force produced by the drive mechanism 24 can urge the lancet 20 to the pricking position as shown in FIG. 6. Potential energy previously stored in the drive mechanism 24 (as discussed above in reference to FIG. 5) has been converted to kinetic energy; i.e., movement of the lancet 20 toward the pricking position shown in FIG. 6.

In the pricking position as shown in FIG. 6, the needle 26 projects from the lancing device 40, enabling the needle 26 to puncture the skin of a user that has placed his finger, for example, against the opening of the lancet receptacle 42. The geometry and material properties of the lancet 20 and other considerations such as the location of the latch 46 can be altered such that a predetermined length of the needle 26 projects from the lancing device 40 when the lancet is in the pricking position and such that the lancet 26 has a predetermined velocity when the needle 26 projects from the lancing device 40.

Additionally, due to the geometry of the drive mechanism 24, the drive mechanism 24 can engage the latch 46 as the lancet 20 is driven toward the pricking position. After the drive mechanism 24 engages the latch 46, momentum of the lancet 20 can deform the drive mechanism 24 as shown in FIG. 6 relative to its normal shape shown in, for example, FIG. 2. The deformation of the drive mechanism 24 can create a force in the axial direction of the lancing device 40, thereby pulling the drive mechanism 24 away from the pricking position toward a retracted position (i.e., a position of the lancet 20 in which the needle 26 is within the lancing device 40 after the lancet 20 has been in the pricking position) as shown in FIG. 7. When the drive mechanism 24 initially becomes deformed due to engagement with the latch 46, the force produced by the drive mechanism 24 may not be sufficient to overcome the momentum of the lancet 20. As such, the lancet 20 continues movement toward the pricking position and the drive mechanism 24 becomes more deformed. Eventually, deformation of the drive mechanism 24 produces a large enough force to overcome the momentum of the lancet body 22, and at this point the drive mechanism 24 urges the lancet body 22 back into the receptacle toward the retracted position. Alternatively, the lancet 20 can remain in the pricking position after actuation of the lancing system 10.

As mentioned above, other examples of lancets can have an alternative geometries from the lancet 20 shown in FIGS. 1-7. FIG. 8 illustrates a lancet 60 including a lancet body 62 and a triangular drive mechanism 64 attached to the lancet body 62. The drive mechanism 64 can be compressed during insertion of the lancet 60 into a lancing device to bias the lancet 60 toward a pricking position. The triangular drive mechanism 64 can be stiffer than the C-shaped drive mechanism 24 discussed above in reference to FIGS. 1-7, and therefore the drive mechanism 64 can produce a greater force than the drive mechanism 24 when compressed the same amount as the drive mechanism 24. The lancet 60 can also include a needle 66 for puncturing the skin of a user and a notch 68 for engagement with a lancing device.

Similarly, FIG. 9 illustrates another lancet 70. The lancet 70 includes a lancet body 72 and an S-shaped drive mechanism 74. The S-shape of the drive mechanism 74 can allow a more secure engagement with a latch in a lancing device than the C-shaped drive mechanism 24 as discussed above in reference to FIGS. 1-7. The lancet 70 can also include a needle 76 for puncturing the skin of a user and a notch 78 for engagement with a lancing device.

In addition, lancing devices with geometries different from the lancing device 40 can be used. For example, FIG. 10 illustrates another lancet 80 for use with a lancing device 50. The lancet 80 can include lancet body 82, a drive mechanism 84 shaped similarly to the drive mechanism 24, and a needle 86 extending from the lancet body 82. Additionally, the lancet 80 can include wings 88. The wings 88 can be angled relative to the axial direction of the lancing device 50. The wings 88 can be resilient such that if bent toward the lancing body 82 (i.e., bent such that the wings 88 are closer to parallel with a longitudinal axis of the lancing device 50), the wings 88 can rebound back toward to their normal shape as shown in FIG. 10. The wings 88 can be formed integrally with the lancet body 82, for example by molding the lancet body 82 and wings 88 in a single mold.

The lancing device 50 can define a lancet receptacle 52 and include two sets of angled indentations 56 and 58, respectively. The diameter of the lancet receptacle 52 can be slightly greater than the diameter of the lancet body 82, but less than the distance between the tips of the wings 88 in their normal, unbiased position. Triggers 54 can be biased to extend into the receptacle 52 from the second set of indentations 58 when actuated.

In operation, the lancet 80 can be inserted into the lancet receptacle 52. During insertion of the lancet 80, the wings 88 can be bent toward lancet body 82 due to contact with the lancing device 50. The lancet 80 can be slid into the lancing device 50 until the drive mechanism 84 is deformed against the lancing device 50 and the wings 88 are aligned with the second set of indentations 58. Once aligned with the indentations 58, the wings 88 can rebound to their normal shape to engage the indentations 58. As a result, though the drive mechanism 84 is deformed and produces a force urging the lancet 80 toward the pricking position, the engagement of the wings 88 and the indentations 58 can retain the lancet 80 in the retained position.

Actuation of the trigger 54 can deflect the wings 88 toward the lancet body 82 to disengage the wings 88 from the indentations 58. Without the engagement between the wings 88 and the indentations 58 to retain the lancet 80, the force produced by drive mechanism 84 can move the lancet 80 toward the pricking position. The first set of indentations 56 can limit the movement of the lancet 80 to prevent the lancet 80 from being ejected from the lancing device 50.

While the above examples illustrate each lancet body and its drive mechanism as being integrally formed, a lancet body and drive mechanism can be formed separately and then attached. For example, as shown in FIG. 11, a lancet 90 includes a lancet body 92 and a separate drive mechanism, shown as a coil spring 94. The lancet body 92 can define a groove 95 and include a needle 96 extending from an opposite end of the body 92 as the groove 95. The body 92 can additionally define a notch 98. The coil spring 94 can be snap-fit onto the groove 95 for attachment to the lancet body 92. The lancet 90 can then be inserted in a lancing device for use in a manner similar to the lancet 24 as described above in reference to FIGS. 1-7. While the separate drive mechanism is shown as the coil spring 94, the drive mechanism can be a different structure such as a leaf spring, or a resilient C-shaped drive mechanism formed separately from a lancet body. The coil spring 94 can be formed of, for example, a metal, a polymer, or a composite. Additionally, the separate drive mechanism 94 can be attached to the lancet body 92 other than with a snap-fit engagement. For example, the drive mechanism 94 can be adhered, clipped, or otherwise attached to the lancet body 92.

Additionally, while the drive mechanism 24 of the lancet 20 can function as both a drive mechanism and a retraction mechanism (i.e., the drive mechanism can bias the lancet 20 from the pricking position to the retracted position), FIG. 12 illustrates a lancet 100 having a separate drive mechanism and retraction mechanism. That is, the lancet 100 includes a drive spring 104 and a retraction spring 107. The drive spring 104 can be attached to a lancet body 102 by way of a snap-fit engagement with a groove 105 defined by the lancet body 102. The retraction spring 107 can be inserted over a needle 106 and clipped to the lancet body 102. The lancet 100 can also define a notch 108 for engagement with a lancing device. Positioning the lancet 100 in the retained position in a lancing device can energize the drive spring 104 to bias the lancet 100 toward the pricking position. Movement of the lancet 100 toward the pricking position can energize the retraction spring 107 to bias the lancet 100 from the pricking position toward the retracted position. Also, a lancet need not have a retraction mechanism attached thereto. Instead, a retraction mechanism can be integral with the lancing device, or the entire lancet system can not include a retraction mechanism.

The above-mentioned embodiments have been described in order to allow easy understanding of the present invention. The invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A lancet for use with a lancing device, the lancet comprising:
a lancet body;
a drive mechanism attached to the lancet body; and
a needle projecting from an end of the lancet body;
wherein the drive mechanism is operative in response to insertion of the lancet in the lancing device to bias the lancet toward a pricking position.

2. The lancet of claim 1, wherein the drive mechanism is energized when the lancet is in a retained position within the lancing device.

3. The lancet of claim 1, wherein the drive mechanism is further operative in response to movement of the lancet toward the pricking position to bias the lancet toward a retracted position.

4. The lancet of claim 1, further comprising a retraction mechanism coupled to the lancing body;
wherein the retraction mechanism is operative in response to movement of the lancet toward the pricking position to bias the lancet toward a retracted position.

5. The lancet of claim 1, wherein the lancet body and drive mechanism are integrally formed.

6. A lancing system comprising:
a lancing device defining a lancet receptacle; and
a lancet slidably received in the lancet receptacle, the lancet including a lancet body having a drive mechanism attached thereto and a needle projecting from an end of the lancet body;
wherein the drive mechanism is operative in response to insertion of the lancet in the lancet receptacle to bias the lancet toward a pricking position.

7. The lancing system of claim 6, wherein the lancing device includes a lancet holder engaged with the lancet in a retained position and a trigger in communication with the lancet holder for selectively disengaging the lancet holder from the lancet.

8. The lancet of claim 6, wherein the drive mechanism is energized when the lancet is in a retained position.

9. The lancet of claim 6, wherein the drive mechanism is further operative in response to movement of the lancet toward the pricking position to bias the lancet toward a retracted position.

10. The lancet of claim 9, wherein the lancing device includes a latch engaged with the drive mechanism when the drive mechanism is in the pricking position.

11. The lancet of claim 6, further comprising a second drive mechanism attached to the lancing body;
wherein the second drive mechanism is operative in response to movement of the lancet toward the pricking position to bias the lancet toward a retracted position.

12. The lancet of claim 6, wherein the lancet body and drive mechanism are integrally formed.

13. A method of using a lancing device and a separate lancet having a needle, the method comprising:
inserting the lancet into the lancing device to energize a resilient portion of the lancet; and
releasing the lancet to allow the lancet to move under the urging of the resilient portion toward a pricking position.

14. The method of claim 13, further comprising:
after inserting the lancet into the lancing device, retaining the lancet in a retained position by engaging a lancet holder with the lancet.

15. The method of claim 14, wherein releasing the lancet includes disengaging the lancet holder from the lancet.

16. The method of claim 13, further comprising:
after releasing the lancet, limiting the movement of the lancet by engaging the lancet with a latch in the lancing device.

17. The method of claim 13, further comprising:
after releasing the lancet, generating a retraction force urging the lancet into the lancing device.

18. The method of claim 17, wherein generating a retraction force includes energizing the resilient portion of the lancet.

19. The method of claim 17, wherein generating a retraction force includes actuating a retraction mechanism attached to the lancet.

20. The method of claim 13, wherein inserting the lancet into the lancing device includes compressing the resilient portion of the lancet.
